(19) United States
(12) Patent Application Publication
(10) Pub. No.: US 2025/0281324 A1
(43) Pub. Date: Sep. 11, 2025

(54) SYSTEM AND METHOD FOR SKIN TEMPERATURE REGULATION

(71) Applicant: BARC Innovations, Inc., Ventura, CA (US)

(72) Inventors: Constanze Rayhrer, Ventura, CA (US); Benno Rayhrer, Ventura, CA (US)

(21) Appl. No.: 18/859,992

(22) PCT Filed: Apr. 19, 2023
(86) PCT No.: PCT/US23/19136

§ 371 Date: Oct. 24, 2024

Related U.S. Application Data
(60) Provisional application No. 63/334,385, filed on Apr. 25, 2022.

(51) Int. Cl. A61F 7/00; A61F 7/02

(52) U.S. Cl. CPC A61F 7/0053; A61F 7/0085; A61F 2007/0045; A61F 2007/006; A61F 2007/0064; A61F 2007/0075; A61F 2007/0086; A61F 2007/0096; A61F 2007/0252

(57) ABSTRACT

A system for regulating skin temperature includes an insulated unit including at least one opening for receiving an appendage of a user, a controller for setting a target internal temperature of the insulated unit, a temperature probe for measuring an actual internal temperature of the insulated unit, and a thermoelectric unit for providing at least one of heated and cooled air into the insulated unit for at least one of raising and lowering the actual internal temperature of the insulated unit. The controller is configured to control power and voltage provided to the thermoelectric unit to maintain the actual internal temperature within the target internal temperature range, such that the skin temperature of the appendage in the insulated unit is regulated within the target internal temperature range while providing medical treatment to the user.

### CROSS-REFERENCE TO RELATED APPLICATION

This application claims priority to U.S. Provisional Application 63/334,385 filed on Apr. 25, 2022, the contents of which is incorporated herein by references in its entirety.

### TECHNICAL FIELD

This disclosure relates generally to an appliance for regulating and maintaining a constant skin temperature.

### BACKGROUND

Every year, over 140,000 men, women, and children in the United States receive chemotherapy as a treatment for cancer. While chemotherapy is designed to kill fast-growing cancerous cells, healthy cells including nerves may often be damaged in the process. As a result, this damage may lead to neuropathy, damage to the sensory nerves in the hands and feet that can cause pain, tingling, and numbness. For many patients, neuropathy may be permanent and can prevent precision movements, return to work, and interfere with other activities of daily living.

Cooling of the hands and feet during chemotherapy administration, often referred to as icing, may prevent permanent damage in a large percentage of patients. Icing may be effective in preventing neuropathy by causing vasoconstriction or cold induced narrowing of the arteries to the skin and nerves. Some studies have also indicated a direct effect on the cellular metabolism, making the individual cells resistant to the side effects of medications. This may be similar to the cooling effect that can allow someone accidentally submerged in ice water to survive longer by slowing the metabolism.

Currently, icing may occur in a variety of methods. Some facilities or patients use ice packs, frozen bags, or submerge their hands and feet in ice water to experience the effect of icing. These methods may cause pain and are messy due to melting ice. The administration of chemotherapy may last up to six hours. As icing is typically done for the duration of chemotherapy, it may also cause frost bite in individuals. Frostbite can also cause neuropathy and can lead to additional tissue loss if severe.

Therefore, a need exists for an electrical cooling platform that provides a safe, painless experience to keep the digits within a therapeutic temperature range without the risk for frostbite. Additionally, a need exists for a system without the mess of ice or water. Hands and feet should be easily removed and slipped back into the device for better convenience and user experience during treatment, for example, including for trips to the restroom, to eat snacks, or to use mobile devices.

### BRIEF DESCRIPTION

According to the disclosure, a system for regulating skin temperature includes an insulated unit including at least one opening for receiving an appendage of a user, a temperature probe for measuring an internal temperature of the insulated unit, a thermoelectric unit for providing at least one of heated and cooled air into the insulated unit; and a controller for maintaining the internal temperature of the insulated unit for regulating skin temperature of the appendage of the user such that the skin temperature is within a therapeutic range for providing medical treatment to the user.

Also according to the disclosure, a method of regulating skin temperature includes inserting an appendage of a user into at least one opening of an insulated unit, setting a target internal temperature range of the insulated unit with a controller, measuring an actual internal temperature of the insulated unit with a temperature probe, providing at least one of heated and cooled air into the insulated unit via at least one thermoelectric unit and at least one fan to at least one of raise or lower the actual internal temperature, and controlling the power and voltage to the thermoelectric unit via the controller to maintain the actual internal temperature within the target internal temperature range.

According to the disclosure, a system for regulating skin temperature includes an insulated unit including at least one opening for receiving an appendage of a user, a controller for setting a target internal temperature of the insulated unit, a temperature probe for measuring an actual internal temperature of the insulated unit, and a thermoelectric unit for providing at least one of heated and cooled air into the insulated unit for at least one of raising and lowering the actual internal temperature of the insulated unit. The controller is configured to control power and voltage provided to the thermoelectric unit to maintain the actual internal temperature within the target internal temperature range, such that the skin temperature of the appendage in the insulated unit is regulated within the target internal temperature range while providing medical treatment to the user.

### BRIEF DESCRIPTION OF THE DRAWINGS

FIG. 1A is a schematic view generally illustrating an outer view of an embodiment of a device used in connection with heating or cooling feet of a user.

FIG. 1B is a schematic view generally illustrating a cross sectional view of an embodiment of a device used in connection with heating or cooling feet of a user.

FIG. 1C is a schematic view generally illustrating the base of an embodiment of a device used in connection with heating or cooling feet of a user.

FIG. 1D is a schematic view generally illustrating a side view of two connected units of an embodiment of a device used in connection with heating or cooling feet of a user.

FIG. 2 is a schematic view generally illustrating an exemplary thermoelectric unit.

FIG. 3A is an external view generally illustrating a front view of an embodiment of a device used in connection with heating or cooling feet of a user.

FIG. 3B is an external view generally illustrating a side view of an embodiment of a device used in connection with heating or cooling feet of a user.

FIG. 3C is an external view generally illustrating a perspective view of an embodiment of a device used in connection with heating or cooling feet of a user.

FIG. 3D is an external view generally illustrating an embodiment of a control panel on a device used in connection with heating or cooling feet of a user.

FIG. 3E is an external view generally illustrating a perspective view of an embodiment of a device while in use by a user.

FIG. 4A is an internal view generally illustrating a side view of an embodiment of a device used in connection with heating or cooling feet of a user.

FIG. 4B is an internal view generally illustrating a perspective view of an embodiment of a device used in connection with heating or cooling feet of a user.

FIG. 5A is an external view generally illustrating a perspective view of an embodiment of a device used in connection with heating or cooling hands of a user with a viewing screen.

FIG. 5B is external view generally illustrating a perspective view of an embodiment of a device used in connection with heating or cooling hands of a user.

FIG. 6 is an exploded view generally illustrating an embodiment of a device used in connection with heating or cooling hands of a user.

FIG. 7 is an exemplary method of regulating skin temperature of a user with a device.

### DESCRIPTION

Referring now to the discussion that follows and the drawings, illustrative approaches to the disclosed systems and methods are described in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive, otherwise limit, or restrict the claims to the precise forms and configurations shown in the drawings and disclosed in the following detailed description.

This disclosure relates generally to a device that maintains a consistent skin temperature of a user's appendages. The device may be used while a user is actively partaking in chemotherapy treatment to prevent chemotherapy side-effects such as neuropathy and nail damage. The device is a small, portable, lightweight, and reusable electric appliance that creates cold air to cool the hands and feet of a user. The device may also be configured to provide warm air to heat the hands and feet of a user. An exemplary device includes a thermoelectric device such as a Peltier unit to create the cooled or heated air. The device may also include a temperature probe and feedback controller to regulate the temperature of the air. Using cold or warm air to regulate the temperature of the hands and feet of a user may maintain a constant skin temperature and provide therapeutic temperature to an appendage while the user receives medical treatment.

Units are built as thermoelectric driven, electrical units. The box is insulated. It is lined with a thin disposable fabric liner to ensure cleanliness and comfort. In an exemplary system, two oval openings provide a way to place appendages such as hands or feet comfortably within the unit. These openings are insulated with a soft sleeve with an elastic closure such as a cinch to prevent air from escaping the unit. When turned on, the heating or cooling rate of the unit is controlled and will not heat or cool more than 3.6° F. (2° C.) per minute to prevent pain to the user. The unit heat and cools to 60° F. with a range of plus or minus 5° F., keeping the skin temperature of the appendage in a therapeutic range for neuropathy prevention and avoiding temperatures known to cause frostbite with prolonged duration. The temperature of the skin is measured based on an external temperature of the skin on the appendage, however in maintaining the external temperature, the temperature throughout the layers of skin are also regulated. The unit includes a safety shut off below 50° F. and above 70° F. in order to prevent burning or freezing of the user's skin.

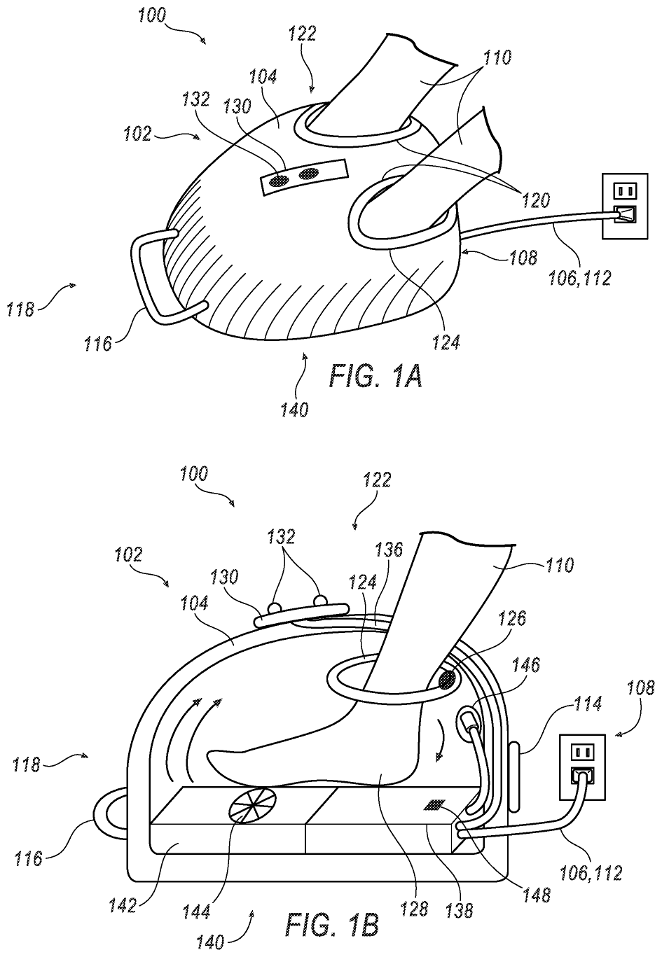

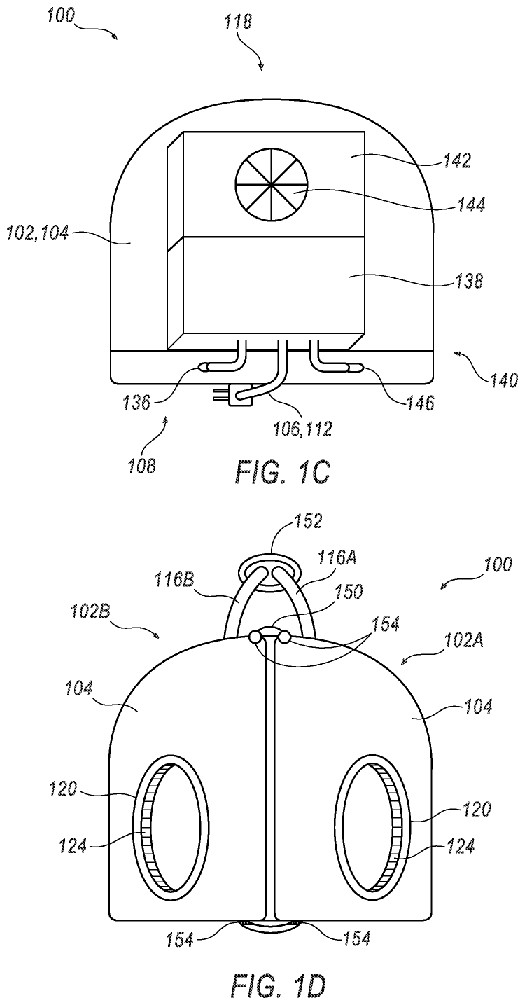

Referring now to the figures, FIGS. 1A-1D illustrate a system 100 for regulating skin temperature in an exemplary configuration for regulating temperature of feet of a user, however other configurations may be used for regulating temperature of different parts of the body, for example, hands of a user. The system 100 is a device 102 for regulating and maintaining a constant skin temperature. Device 102 includes an insulated unit 104 in which an appendage 110 is inserted into and in which temperature is cooled or heated and maintained. Device 102 is an electric device having an external power supply 106, such as a power cord 112 and plug extending from a posterior end 108 of device 102. Power cord 112 may be, in some instances, longer than eight feet long to ensure device 102 may be plugged into a power source behind a chair used in chemotherapy or other medical treatments. Insulated unit 104 may include a storage compartment 114, or in instances, a wrapping mechanism, for management of power cord 112 to provide compact storage and portability when device 102 is not in use. Storage compartment 114 may be positioned on posterior end 108 of unit 104 in proximity to power supply 106. In other examples, device 102 may be battery operated to provide therapeutic heating or cooling to a user where a external power supply is not available.

Insulated unit 104 may include a handle 116, for example, on an anterior end 118. Handle 116 is a u-shaped type for comfortability and ergonomic use by a user. Handle 116 may be of other shapes and sizes, but should remain comfortable for user who may be experiencing dry skin, rashes, and pain from, for example, chemotherapy. Handle 116 may be foldable, for example, into a storage compartment, to increase compactness of insulated unit 104 for storage and travel.

In an exemplary configuration as illustrated in FIGS. 1A-1D, insulated unit 104 includes openings 120 on a top surface 122 for insertion of a user's appendages 110, for example feet, into insulated unit 104. In exemplary configurations for regulating temperature of hands, for example, openings 120 may be located on a posterior end 108 for ease of appendage 110 insertion. Openings 110 are insulated, oval apertures in surface (e.g., top surface 122, posterior end 108) of insulated unit 104 for providing a location to place targeted appendage 110 comfortably into device 102. Openings 110 may include a sleeve 124 with a closing device 126 such as an elastic closure or a cinch. Sleeve 124 provides comfort to a user to ensure appendage 110 is in contact with a soft surface rather than a hard exterior cover of unit 104. Closing device 126 tightens sleeve 124 around appendage 110 to prevent air from escaping from insulated unit 104 while in use and the closing device 126 is pulled tight. Openings 110 are further lined with a disposable liner 128 configured to provide comfort and further insulation to user, as well as to ensure sanitation amongst multiple users of device as liners 128 may be replaced between device 102 uses.

Device 102 includes a control panel 130 for use by a user or, for example, a medical practitioner providing medical service to the user. In the exemplary configuration of FIGS. 1A-1D, control panel 130 is located on a top surface 120 for ease of use by a user bending down to operate device 102 while feet are positioned in openings 110. In other examples, control panel 130 may be located on other surfaces (e.g., posterior end 108 or a side surface). Control panel 130 includes buttons 132 for, for example, turning power on to device 102 and for controlling temperature. Control panel 130 may include additional operations including, but not limited to, USB power ports 134 for connecting Bluetooth connection devices such as touchpads or a mouse for use with a laptop, cell phone, or other external electronic devices. Control panel cord 136 connects control panel 130 to a controller 138 for operation of device 102.

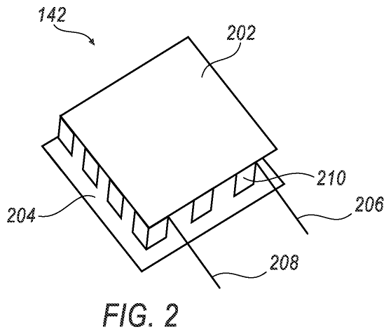

Positioned inside insulated unit 104 and towards a bottom surface 140 is a thermoelectric unit 142, which in examples, may be a Peltier unit. An exemplary thermoelectric unit is illustrated in FIG. 2. Thermoelectric unit 142 includes two ceramic plates 202, 204, separated by semiconductor pellets 206, 208. A first ceramic plate 202 absorbs heat, while a second ceramic plate 204 dissipates heat when a current is passed through a first semiconductor pellet 206 and a second semiconductor pellet 208. In an embodiment of device 2, current is passed to first ceramic plate 202 that absorbs heat such that thermoelectric unit 142 is a cooling unit. In another embodiment, current direction may be modified and reversed such that current is provided to second ceramic plate 204 that dissipates heat such that thermoelectric unit 142 is a heating unit. Thermoelectric unit 142 includes at least one set of ceramic plates 202, 204 and semiconductor pellets 206, 208, but may include additional sets for increased heating and cooling power.

Thermoelectric unit 142 includes an at least first fan 144. First fan 144 is driven by electricity to cycle cooled air from thermoelectric unit 142 into and throughout insulated unit 142. Fan 144 runs continuously while device 102 is powered to provide a white noise to user to not disturb users and individuals within a close proximity to device 102 and to ensure rotation of air inside device 102 and accurate temperature control. For example, fan 144 mixes air throughout device 102 such that the internal temperature remains consistent throughout device 102 and hot spots or cool spots are prevented and/or minimized. Thermoelectric unit 142 is positioned adjacent to controller 138 and is provided with information from controller 138 to determine when to operate thermoelectric unit 142 and in which operation (e.g., heating operation or cooling operation) thermoelectric unit 142 should operate in to stay in a target temperature range. A temperature probe 146 is connected to controller 138 for measuring and monitoring an internal temperature of insulated box 104. Temperature probe 146 is configured to work in conjunction with controller 138, communicating the internal temperature to controller 138 such that controller 138 can provide operational controls to thermoelectric unit 142 to produce air of a necessary temperature to maintain target temperature range. Temperature probe 146 may additional include a skin temperature probe for measuring an external skin temperature of the user compared to internal temperature of insulated unit 104.

Controller 138 and thermoelectric unit 142 are configured to operate in conjunction with each other such that thermoelectric unit 142 operates based on information received from controller 138. In an exemplary configuration, thermoelectric unit 142 is configured to heat or cool at a controlled rate and will not heat or cool more than 3.6° F. per minute to prevent pain to a user from rapid cooling and/or heating. In an exemplary setting, thermoelectric unit 142 cools or heats to approximately 60° F., measured by temperature probe 146. Thermoelectric unit 142 may heat or cool to a range of plus or minus 5° F. from 60° F. to keep appendage 110 temperature within a target therapeutic range for neuropathy prevention and avoiding temperatures known to cause frostbite with prolonged duration. Controller 138 includes a safety shut off 148 configured to turn power to thermoelectric unit 142 off when temperature probe 146 measures below 50° F. or above 70° F.

Illustrated in FIG. 1C is an exemplary configuration of device 102 viewed from bottom surface 140. Device 102 includes thermoelectric unit 142 and controller 138 located proximate the bottom of insulated unit 104. Extending outward from insulated unit 104 on posterior end 108 is power supply 106, 108 for providing power to device 102. Extending from controller 138 and located in insulated unit 104 is temperature probe 146 for measuring temperature inside insulated unit 104. Extending from controller 138 and built into walls of insulated unit 104 is control panel cord 136 for controlling controller 138 based on received inputs from control panel 130.

Illustrated in FIG. 1D is an exemplary configuration of a first device 102A and a second device 102B connected to each other for portability of more than one device 102 and viewed from a side perspective. In the exemplary configuration, first device 102A and second device 102B are placed adjacent to each other at their bottom surfaces 140. A first strap 148 may connect first device 102A to second device 102B at a posterior end 108 of each of the devices. For example, first strap 148 may be connected to each of first device 102A and second device 102B via connector 154, for example, a snap fastener or a buckle. A second strap 150 may connect first device 102A to second device 102B at an anterior end 118 of each device. For example, second strap 150 may be connected to each of first device 102A and second device 102B via connector 154. Connecting first device 102A and second device 102B is provided for ease of a user's ability to travel with multiple devices 102. On anterior end 118, a handle strap 152 may attach first handle 116A from first device 102A to second handle 116B from second device 102B so a user may conveniently hold handles 116A, 116B from both devices 102A, 102B concurrently.

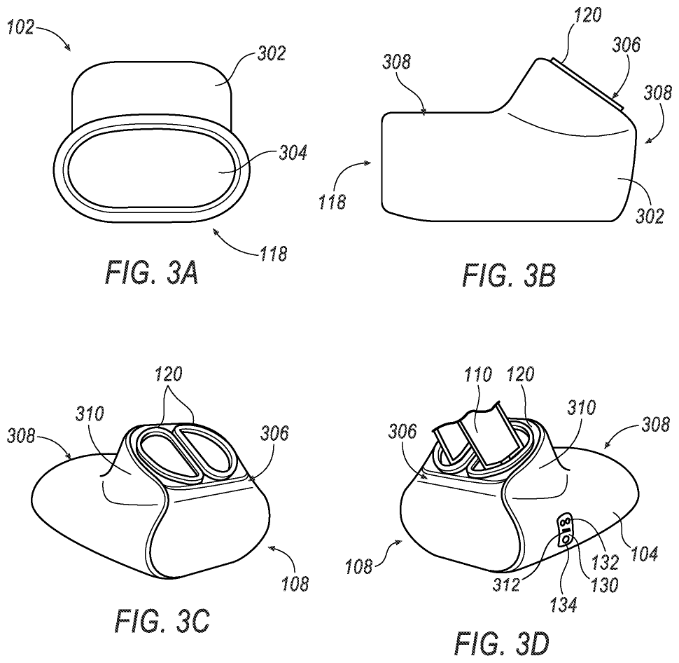

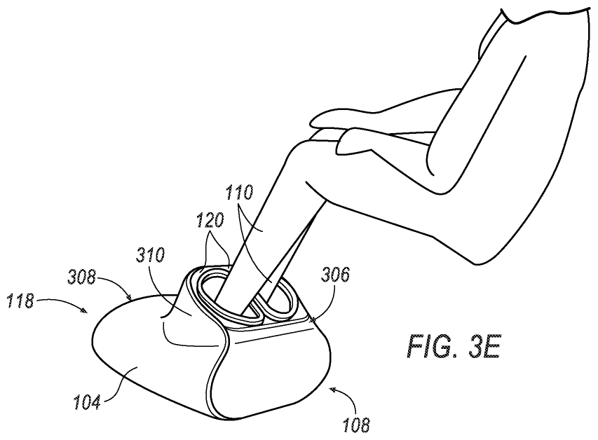

Illustrated in FIGS. 3A-3E is an exemplary embodiment of device 102 configured to be used for regulating and maintaining temperature of feet of a user. FIG. 3A illustrates device 102 from anterior end 118. Insulated box 104 is surrounded by an external cover 302 that provides protection and concealment of the internal electronics. External cover 302 may include a venting grill 304, for example, on anterior end 118 of device 102. Grill 304 is designed to expel air from within insulated unit 104 such as a warm air exhaust.

Referring to FIG. 3B, an exemplary embodiment of device 102 is illustrated from a side view. Insulated unit 104 includes a user engaging portion 306 at posterior end 108 and a covered portion 308 at anterior end 118. User engaging portion 306 includes openings 120 for a user to insert appendages 110 into insulated unit 104. In embodiments, user engaging portion 306 may be angled at a slightly upward angle for user comfortability (i.e., for a user to insert feet into openings 120 while sitting in a chair). In additional embodiments, user engaging portion 306 may be relatively flat for user comfortability (i.e., for a user to slide hands into openings 120 when sitting at a table).

Referring to FIG. 3C, an exemplary embodiment of device 102 is illustrated from a perspective view, generally facing a first side and posterior end 108. User engaging portion 306 includes a raised protrusion 310 from external cover 302 to provide openings 110 at an upward angle to user. FIG. 3D illustrates an exemplary embodiment of device 102 illustrated from a perspective view, generally facing a second side and posterior end 108. In embodiments, control panel 130 may be positioned towards posterior end 108 of device 102. Control panel 130 includes buttons 132 for user to input feedback and controls regarding, for example, power and temperature selection. Additionally, control panel 130 may include, for example, USB ports 134 and auxiliary cord ports 312 for connection and use of Bluetooth devices such as computer mice or touchpads. FIG. 3E illustrated an exemplary embodiment of device 102 illustrated from a perspective view, generally facing a first side and posterior end 108, while in use by a user. User is illustrated sitting with appendages 110 (i.e., feet) inserted into openings 120 of insulated unit 104.

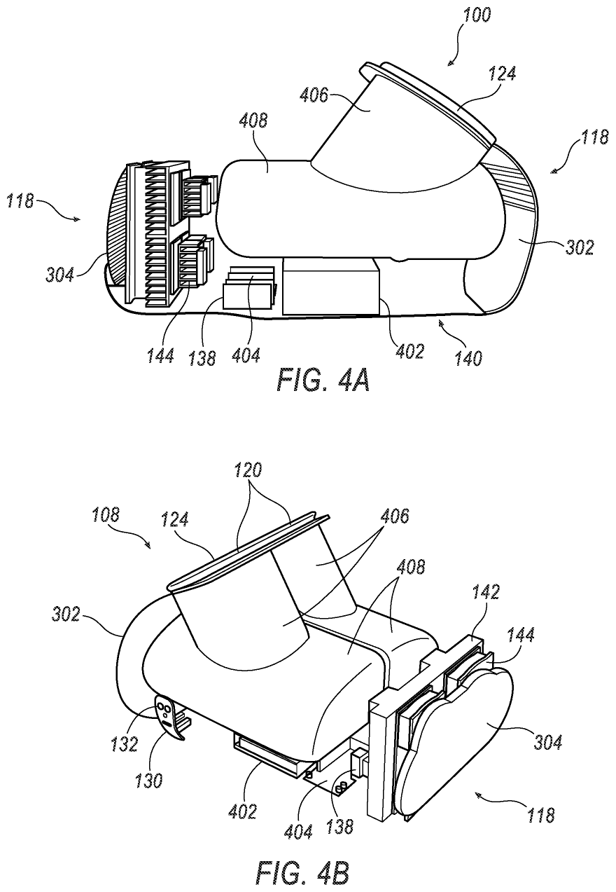

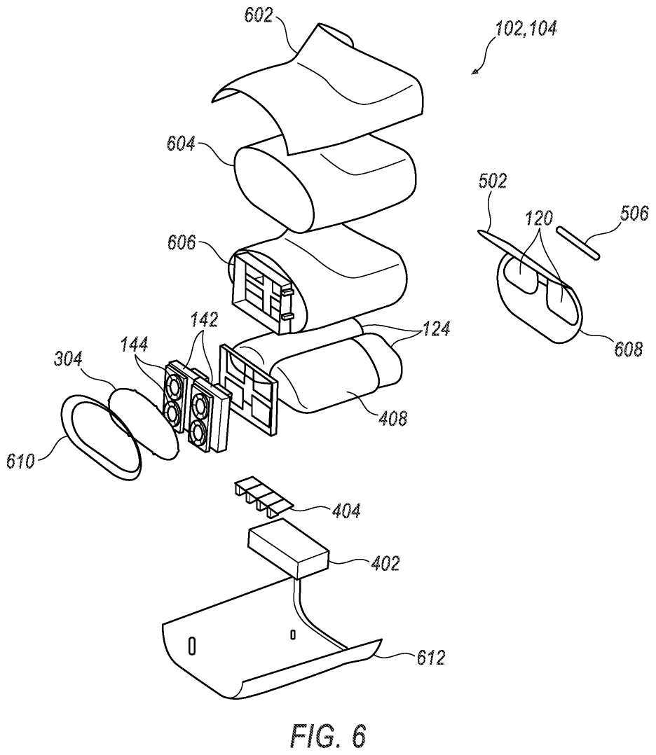

As illustrated in FIGS. 4A and 4B, an exemplary configuration of device 102 is illustrated from an internal view. Openings 120 include sleeve 124 around outer rim of opening 120 to provide comfort to appendage 110 when inserted in opening 120. Openings 120 provide access to insulated bag 408 for appendage 110. Insulated bags 408 may include an angled portion 406 which connects insulated bag 408 to opening 120 in configurations with raised protrusion 310 (i.e., covering portion of a user's ankle and legs when sitting with feet in bags 408). Bags 408 may be made of a substantially thin fabric material or non-weaving fabric using a cut-and-sew process. A foam outer later (as illustrated in FIG. 6) provides insulation to bag 408. Insulated bags may additionally be lined internally with liner 128 (e.g., a disposable liner) for sanitation between uses such as where devices 102 are rented or used in a facility with multiple patients.

Referring to FIG. 4A, device 102 illustrates a side view of the internal components of device 102. Electricity received from power supply 106 is provided to a power adapter 402 and power boards 404 for operating device 102. Power adapter 402 and power boards 404 may be positioned adjacent bottom surface 140 of device 102 to position weight of power adapter 402 and power board 404 towards a lower surface. Power adapter 402 and power board 404 are used to provide power to and operate controller 138, temperature probe 146, and thermoelectric unit 142 based on input from control panel 130.

Referring to FIG. 4B, device 102 illustrates a perspective view, generally from a side and anterior end, of the internal components of device 102. Thermoelectric unit 142 may be positioned toward anterior end 118 of insulated unit 104 to save space in a Y-dimension of insulated unit 104, as well as being positioned close to venting grill 304 to allow venting of exhaust waste. Control panel 130 may be positioned on a side surface toward posterior end 108 for easy access to a user.

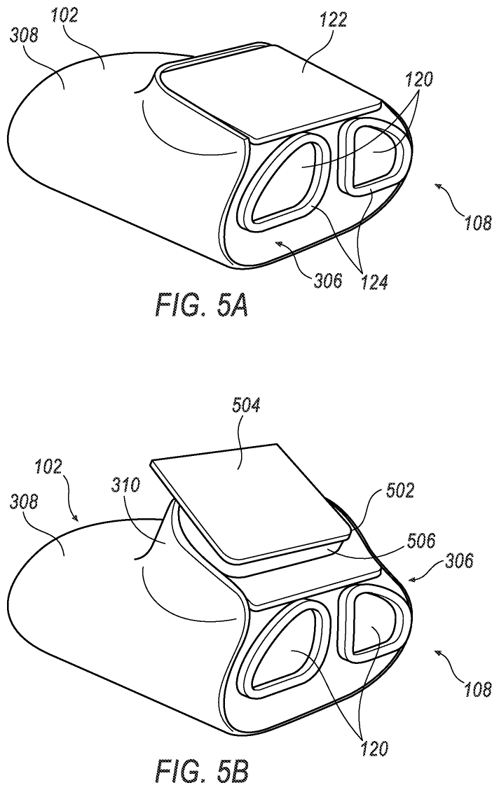

An exemplary configuration of device 102 configured to regulate or maintain temperature of a user's hands is illustrated in FIGS. 5A and 5B. In embodiments, as illustrated in FIG. 5A, device 102 may include a generally flat top surface 122. In embodiments, as illustrated in FIG. 5B, device 102 may include a raised protrusion 310 on top surface 122. As openings 120 are below raised protrusion 310 in the illustrated embodiment, raised protrusion 310 is provided as an angled surface for facing a user's face (i.e., for placing reading or electronic materials for user viewing).

As illustrated in FIG. 5A, device 102 has a generally flat top surface 122. User engaging portion 306 is at posterior end 108, such that openings 120 are generally on posterior end 108 facing surface. Openings 120 are configured such that device 102 may be placed on a surface, for example a table, and a user can slide their appendage (i.e., their hands) into openings 120 and lay their arms flat on the surface and/or table. As illustrated in FIG. 5B, device 102 may include raised portion 310 on top surface 122 of external cover 302. In embodiments as illustrated in FIG. 5B, openings 120 are generally on posterior end 108 facing surface in a substantially flat configuration. Raised portion 310 is positioned above openings 120 such that raised portion 310 may provide a viewing surface 510 to a user. Viewing surface 510 may be generally angled upward toward a user's face such that viewing materials, for example a book or connected device 504, may rest on viewing surface and be visible to a user to engage with while using device 102. A ledge 506 is provided near a bottom of viewing surface 502 to provide a surface for viewing materials 504 to rest on when positioned on viewing surface 502.

FIG. 6 illustrates an exploded view of an embodiment of device 102 configured for cooling or heating appendages (i.e., hands) of a user. Insulated unit 104 includes several layers to ensure proper insulation for higher effectiveness due to loss of cool and/or hot air and temperature maintenance. External cover 302 of insulated box 104 includes a top cover 602 and a bottom cover 614 nestled together such that top cover 602 and bottom cover 614 make up substantially top surface 122, bottom surface 140, and both side surfaces of external cover 302. A back cover 608 is provided to make up substantially a posterior end 108 facing surface of external cover 302. Venting grill 304 is encompassed in a grill frame 610. The combined venting grill 304 in grill frame 610 makes up anterior end 118 facing surface of external cover.

Within external cover 310 components, a foam layer 604 is generally positioned adjacent to external cover 310 in an oval tube shape. Foam layer 604 is provided to act as a source of insulation to maintain temperature within insulated box 104 to reduce energy production and increase effectiveness of device 102. Located inward of foam layer 604 is an inner frame 606 to provide structure to internal components of device 102 such as the thermoelectric unit 142 and insulated bags 408. Insulated bags 408 are positioned inside inner frame 606. On an anterior end 118 facing side of inner frame, a thermoelectric unit frame 614 is included to house the at least one thermoelectric unit 142 and the at least one fan 144. Venting grill 304 and grill frame 610 are positioned generally anterior to inner frame 606 and thermoelectric unit 138. Power adapter 402 and power boards 404 are positioned generally adjacent to bottom cover 402 to distribute power assembly weight towards bottom surface 140. Back cover 608 includes viewing surface 502 and ledge 506 in illustrated embodiment. Openings 120 are included in back cover 120. Sleeves 124 are included in insulated bags 408 and extend out of openings 120 to provide comfortable edge to openings 120 for user.

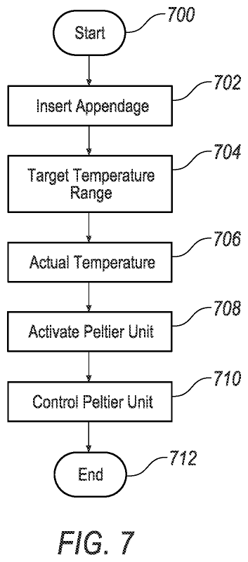

Referring now to FIG. 7, an exemplary method of operating heating and/or cooling device 102 to regulate skin temperature of a user is provided. At 700, user begins operating device 102. At 702, user inserts an appendage 110 into insulated unit 104, for example, inserting hands or feet of the user into openings 120 and insulated bags 408 of insulated unit 104. At 704, user operates control panel 130 to power device 102 on and set a target temperature range using buttons 132 on control panel 130. Device 102 may also be Bluetooth enabled and could receive input for power or target temperature range via Bluetooth capable devices. A target temperature range may be, for example, between 55° F. to 65° F. Target temperature range of 55° F. to 65° F. is ideal for keeping appendage temperature in a therapeutic range for neuropathy prevention and avoiding temperatures prone to cause frostbite at prolonged duration. At 706, temperature probe 146 measures an actual internal temperature of insulated unit 104. In embodiments with a skin temperature probe, skin temperature probe may measure actual external skin temperature. At 708, controller 138 compares actual internal temperature and/or actual external skin temperature to the target temperature range. If actual internal temperature and/or actual external skin temperature is not within target temperature range, controller 138 provides power to thermoelectric unit 142 to activate the at least one thermoelectric unit 142 and at least one fan 144. Power and voltage are provided to thermoelectric unit 142 to provide heated or cooled air to insulated unit 104. For example, if actual temperature is above target temperature range, voltage will be provided to thermoelectric unit 142 to provide cooled air to insulated unit 104 to decrease actual temperature. If actual temperature is below target temperature unit, voltage is reversed and thermoelectric unit 142 will provide heated air to insulated unit 104 to raise actual temperature. Controller 138 controls thermoelectric unit 142 to ensure actual temperature is not increased or decreased by a change of more than 3.9° F. per minute to prevent pain for fast cooling or heating to skin on appendage 110. At 710, controller 138 continues to maintain control of thermoelectric unit 142 by increasing or reducing power and/or reversing voltage to thermoelectric unit 142 to maintain the actual temperature within the target temperature range. Controller 138 will continue to maintain temperature until 712, when user ends operation of device 102 by shutting device 102 off or when a safety shut off pulls power from device 102 if actual temperature reaches an unsafe lever, for example, below 50° F. or above 70° F.

The disclosed technology may be configured into a therapy helmet for preventing hair loss during chemotherapy. For example, a device 102 may be configured into a helmet design similar to a free-standing hair dryer for providing controlled heating or cooling of the scalp of a user during administration of chemotherapeutic agents that cause hair loss. A helmet design utilizes continuous skin temperature monitoring to assure maintenance of therapeutic temperature range and frostbite protection. This scalp device utilizes circulating air and continuous skin temperature monitoring to assure efficacy and prevent skin damage.

The disclosed technology may also be configured as a heating or cooling device for physical therapy, postoperative cooling, and for treatment of sports injuries. For example, the device 102 may be configured to include insulated bags 408 which conform to body contours and provides controlled heating or cooling, or heating alternative with cooling, for a variety of skeletal or muscular therapies. Skin temperature feedback from temperature probe 146 will assure the device is operating in a therapeutic range and maximum and minimum temperature controls will prevent burns or frost bite to vulnerable tissues. The disclosed device utilizes circulating air and continuous skin temperature monitoring to assure efficacy and prevent skin damage.

The disclosed technology may be modified to be used as a device for sports activities involving hands and feet. The device can be used as designed for controlled cooling after sports activities causing swelling in hands and feet, for example marathon or ultra-runners that treat swelling and contact with hot pavement which can be severe causing blistering, skin loss, and inability to wear shoes. The device utilizes circulating air and continuous skin temperature monitoring to assure efficacy and prevent skin damage.

The disclosed technology may be modified to be used as a heating device for sports activities, hypothermia, Reynaud's Syndrome, or neuropathy. This device can be used as designed as a controlled device for hands and feet by reversing the voltage of the thermoelectric unit to provide warm air. The device temperature control range can measure the skin temperature and adjust to warm the hands and feet by 1-2° F. per minute prevent pain from rewarming. Skin temperature of the hands and feet is usually 80° F.-84° F. at a room temperature of 70° F. Maximum temperature can be set at 105° F. to prevent burns. This application can be used for symptomatic relief of muscle spasm, Reynaud's Syndrome, and neuropathy. Other uses would include rewarming after cold climate sports activities such as skiing, ice skating, hockey, or after industrial work in cold environments. The device may also be used to provide controlled rewarming of hands and feet as a treatment for hypothermia. The device utilizes circulating air and continuous skin temperature monitoring to assure efficacy and prevent skin damage.

Thus, according to the disclosure, a system for regulating skin temperature includes an insulated unit including at least one opening for receiving an appendage of a user, a temperature probe for measuring an internal temperature of the insulated unit, a thermoelectric unit for providing at least one of heated and cooled air into the insulated unit; and a controller for maintaining the internal temperature of the insulated unit for regulating skin temperature of the appendage of the user such that the skin temperature is within a therapeutic range for providing medical treatment to the user.

Also according to the disclosure, a method of regulating skin temperature includes inserting an appendage of a user into at least one opening of an insulated unit, setting a target internal temperature range of the insulated unit with a controller, measuring an actual internal temperature of the insulated unit with a temperature probe, providing at least one of heated and cooled air into the insulated unit via at least one thermoelectric unit and at least one fan to at least one of raise or lower the actual internal temperature, and controlling the power and voltage to the thermoelectric unit via the controller to maintain the actual internal temperature within the target internal temperature range.

According to the disclosure, a system for regulating skin temperature includes an insulated unit including at least one opening for receiving an appendage of a user, a controller for setting a target internal temperature of the insulated unit, a temperature probe for measuring an actual internal temperature of the insulated unit, and a thermoelectric unit for providing at least one of heated and cooled air into the insulated unit for at least one of raising and lowering the actual internal temperature of the insulated unit. The controller is configured to control power and voltage provided to the thermoelectric unit to maintain the actual internal temperature within the target internal temperature range, such that the skin temperature of the appendage in the insulated unit is regulated within the target internal temperature range while providing medical treatment to the user.

When introducing elements of various embodiments of the disclosed materials, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the preceding discussion is generally provided in the context of a heating or cooling device to be used on hands or feet of a chemotherapy patient, it should be appreciated that the present techniques are not limited to such limited contexts. The provision of examples and explanations in such a context is to facilitate explanation by providing instances of implementations and applications. The disclosed approaches may also be utilized in other contexts or configurations such as cold therapy helmets. The device may also be reconfigured to a different temperature range, providing heating or cooling for treatments such as for physical therapy, postoperative cooling, or treatment from sports injuries.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions, or equivalent arrangements not heretofore described, but which are commensurate with the spirit and scope of the disclosed materials. Additionally, while various embodiments have been described, it is to be understood that disclosed aspects may include only some of the described embodiments. Accordingly, that disclosed is not to be seen as limited by the foregoing description but is only limited by the scope of the appended claims.

What is claimed is:

1. A system for regulating skin temperature, comprising:
an insulated unit including at least one opening for receiving an appendage of a user;
a temperature probe for measuring an internal temperature of the insulated unit;
a thermoelectric unit for providing at least one of heated and cooled air into the insulated unit; and
a controller for maintaining the internal temperature of the insulated unit for regulating the skin temperature of the appendage of the user such that the skin temperature is within a therapeutic range for providing medical treatment to the user.

2. The system of claim 1, wherein the controller maintains the internal temperature of the insulated unit within a target internal temperature range between 55° F. to 65° F.

3. The system of claim 1, further including a safety shut off for stopping power to the thermoelectric unit when the internal temperature is one of below 50° F. and above 70° F.

4. The system of claim 1, wherein the thermoelectric unit is configured to provide the at least one of heated and cooled air into the insulated unit such that the internal temperature of the insulated unit does not exceed a change of 2° F. per minute.

5. The system of claim 1, wherein the at least one opening is positioned substantially toward a posterior end of the insulated unit and is configured to receive at least one of hands or feet of the user.

6. The system of claim 5, further including a sleeve surrounding an edge of the at least one opening, the sleeve including a cinch to secure the sleeve to the appendage of the user.

7. The system of claim 1, further including a control panel, the control panel configured to receive input from the user for providing power to the insulated unit and controlling the internal temperature.

8. The system of claim 1, further including at least one fan for cycling air throughout insulated unit for ensuring the internal temperature is substantially consistent throughout the insulated unit.

9. The system of claim 1, wherein the temperature probe includes a skin temperature probe for measuring the external skin temperature of the appendage.

10. The system of claim 1, further comprising a venting grill for expelling exhaust air from insulated unit to maintain the internal temperature of insulated unit.

11. The system of claim 1, further comprising an external cover surrounding the insulated unit; and
wherein the external cover further includes a raised protrusion generally towards the posterior end of the insulated unit.

12. The system of claim 11, wherein the raised protrusion includes a viewing surface and a ledge.

13. The system of claim 11, wherein the raised protrusion includes the at least one opening.

14. The system of claim 1, further including an insulated bag attached to the at least one opening to surround the appendage in the insulated unit.

15. The system of claim 14, further including a disposable liner to line an inner surface of the insulated bag.

16. A method of regulating skin temperature, comprising:
inserting an appendage of a user into an opening of an insulated unit;
setting a target internal temperature range of the insulated unit with a controller;
measuring an actual internal temperature of the insulated unit with a temperature probe;
providing at least one of heated and cooled air into the insulated unit via at least one thermoelectric unit and at least one fan to at least one of raise or lower the actual internal temperature; and controlling the power and voltage to the thermoelectric unit via the controller to maintain the actual internal temperature within the target internal temperature range to regulate skin temperature of the appendage to provide medical treatment to the appendage.

17. The method of claim 16, further comprising maintaining the target internal temperature range between 55° F. to 65° F.

18. The method of claim 16, further comprising at least one of raising or lowering the actual internal temperature no more than 2° F. per minute.

19. The method of claim 16, further comprising shutting off power to the thermoelectric unit if the actual internal temperature is at least one of below 50° F. and above 70° F.

20. A system for regulating skin temperature, comprising:
an insulated unit including at least one opening for receiving an appendage of a user;
a controller for setting a target internal temperature range of the insulated unit;
a temperature probe for measuring an actual internal temperature of the insulated unit; and
a thermoelectric unit for providing at least one of heated and cooled air into the insulated unit for at least one of raising and lowering the actual internal temperature of the insulated unit;
wherein the controller is configured to control power and voltage provided to the thermoelectric unit to maintain the actual internal temperature within the target internal temperature range, such that the skin temperature of the appendage in the insulated unit is regulated within the target internal temperature range while providing medical treatment to the user.
